(19) United States
(12) Patent Application Publication
(10) Pub. No.: US 2024/0287278 A1
(43) Pub. Date: Aug. 29, 2024

(54) METHOD FOR DEPOLYMERISING A POLYESTER FILLER COMPRISING A PRE-MIXING STAGE OF THE FILLER

(71) Applicants: IFP ENERGIES NOUVELLES, RUEIL-MALMAISON (FR); JEPLAN, INC., KAWASAKI CITY (JP)

(72) Inventors: Yacine HAROUN, RUEIL-MALMAISON CEDEX (FR); Cyprien CHARRA, RUEIL-MALMAISON CEDEX (FR)

(73) Assignees: IFP ENERGIES NOUVELLES, RUEIL-MALMAISON (FR); JEPLAN, INC., KAWASAKI CITY (JP)

(21) Appl. No.: 18/571,046

(22) PCT Filed: Jun. 7, 2022
(86) PCT No.: PCT/EP2022/065426

§ 371 Date: Dec. 15, 2023

(30) Foreign Application Priority Data
Jun. 17, 2021 (FR) FR2106437

(51) Int. Cl. C08J 11/24; C07C 67/03

(52) U.S. Cl. CPC C08J 11/24; C07C 67/03; C08J 2367/02

(57) ABSTRACT

The invention relates to a process for depolymerization of a polyester feedstock, comprising:
a) conditioning of the feedstock using a means for at least partially melting the feedstock and at least one mixer, which are fed with the feedstock and a diol stream, with a ratio by weight between the diol stream and the feedstock of between 0.01 and 6.00, the volume degree of dilution with diol in each mixer being between 3% and 70%;
b) depolymerization of the polyester feedstock at 150-300° C., the ratio by weight between the diol and the diester in step b) being adjusted between 0.3 and 8.0;
c) optionally, the separation of the diol, at a temperature of between 60 and 250° C. and a pressure lower than that of step b).

### TECHNICAL FIELD

The invention relates to a process for depolymerization of a polyester, preferably comprising polyethylene terephthalate (PET), to obtain a diester monomer stream, more particularly a stream of bis(2-hydroxyethyl) terephthalate (BHET). More particularly, the invention relates to a process for depolymerization of a polyester feedstock preferably comprising PET, comprising a particular step for conditioning the polyester feedstock by a staged premixing of said feedstock with an alcohol stream, so as to obtain a conditioned feedstock advantageously in the form of a homogeneous mixture, exhibiting a viscosity of less than or equal to 50 mPa·s, which is then sent to the depolymerization reaction unit.

### PRIOR ART

The chemical recycling of polyester, in particular of polyethylene terephthalate (PET), has been a subject of numerous studies aimed at breaking down the polyester, recovered in the form of waste, into monomers which will be able to be used again as feedstock for a polymerization process.

Numerous polyesters result from collection and sorting networks. In particular, polyester, in particular PET, may originate from the collection of bottles, container trays, films, resins and/or fibres composed of polyester (for instance textile fibres, tyre fibres). The polyester resulting from collection and sorting channels is referred to as polyester or PET to be recycled.

PET to be recycled can be classified into four main categories:
clear PET, predominantly consisting of transparent colourless PET (in general at least 60% by weight) and of transparent azure-coloured PET, which does not contain pigments and may be used in mechanical recycling processes,
dark or coloured (green, red, etc.) PET, which can generally contain up to 0.1% by weight of dyes or pigments but remains transparent or translucent;
opaque PET, which contains a significant amount of pigments at contents typically varying between 0.25% and 5.0% by weight in order to opacify the polymer. Opaque PET is increasingly being used, for example in the manufacture of food containers, such as milk bottles, in the composition of cosmetic, plant-protection or dye bottles;
multilayer PET, which comprises layers of polymers other than PET or a layer of recycled PET between layers of virgin PET (that is to say, PET which has not undergone recycling), or a film of aluminium, for example. Multilayer PET is used, after thermoforming, to produce packagings, such as container trays.

The collection channels, which supply the recycling channels, are structured differently from one country to another. They are changing so as to maximize the amount of plastic upgraded from waste as a function of the nature and amount of the streams and of the sorting technologies. The channel for recycling these streams generally consists of a first step of conditioning in the form of flakes during which bales of raw packaging are washed, purified and sorted, ground and then purified again and sorted to produce a stream of flakes generally containing less than 1% by mass of “macroscopic” impurities (glass, metals, other plastics, wood, paper, cardboard, inorganic elements), preferentially less than 0.2% of “macroscopic” impurities and even more preferentially less than 0.05%.

Clear PET flakes may subsequently undergo an extrusion-filtration step to produce extrudates which can subsequently be reused as a mixture with virgin PET to produce new products (bottles, fibres, films). A step of solid state polymerization under vacuum (known by the abbreviation SSP) is necessary for food uses. This type of recycling is known as mechanical recycling.

Dark (or coloured) PET flakes can also be recycled mechanically. However, the colouration of the extrudates formed from the coloured streams limits the uses: dark PET is generally used to produce packaging straps or fibres. The outlets are thus more limited in comparison with those of clear PET.

The presence of opaque PET containing pigments at high contents, in PET to be recycled, presents problems to recyclers since it adversely affects the mechanical properties of recycled PET. Opaque PET is currently collected with coloured PET and is found in the coloured PET stream. In view of the development of the uses for opaque PET, the contents of opaque PET in the stream of coloured PET to be recycled are currently between 5-20% by weight and are tending to increase. In a few years' time, it will be possible to achieve contents of opaque PET in the coloured PET stream of greater than 20-30% by weight. However, it has been shown that, above 10-15% of opaque PET in the coloured PET streams, the mechanical properties of the recycled PET are adversely affected (cf. Impact du développement du PET opaque blanc sur le recyclage des emballages en PET [Impact of the growth of white opaque PET on the recycling of PET packagings], preliminary report of COTREP of May 12, 2013) and prevent recycling in the form of fibres, the main outlet of the channel for coloured PET.

Dyes are natural or synthetic substances which are soluble, in particular in the polyester material, and are used to colour the material into which they are introduced. The dyes generally used have different natures and often contain heteroatoms of O and N type, and conjugated unsaturations, such as, for example, quinone, methine or azo functions, or molecules such as pyrazolone and quinophthalone.

Pigments are finely divided substances which are insoluble, in particular in the polyester material, and which are used to colour and/or opacify the material into which they are introduced. The main pigments used to colour and/or opacify the polyesters, in particular PET, are metal oxides, such as TiO₂, CoAl₂O₄ or Fe₂O₃, silicates, polysulfides and carbon black. The pigments are particles with a size generally of between 0.1 and 10 μm and predominantly between 0.4 and 0.8 μm. The complete removal of these pigments, which is necessary in order to envisage recycling the opaque PET, by filtration is technically difficult as they have an extremely high clogging capability.

The recycling of coloured and opaque PETs is thus extremely problematic.

Patent application US 2006/0074136 describes a process for depolymerization by glycolysis of coloured PET, in particular resulting from the recovery of green-coloured PET bottles. The feedstock treated by this process takes the form of PET flakes and is contacted with ethylene glycol in a reactor at a temperature of between 180 and 280° C. for several hours. The BHET obtained on conclusion of the glycolysis step is purified over activated carbon to separate out certain dyes, such as blue dyes, followed by extraction of the residual dyes, such as the yellow dyes, with an alcohol or with water. The BHET crystallizes in the extraction solvent and is then separated in order to be used in a polymerization process.

In patent application US 2015/0105532, the post-consumer PET, which comprises a mixture of different PETs, such as clear PET and coloured PETs such as blue PET, green PET and/or amber PET, in the form of flakes, is depolymerized by glycolysis in the presence of ethylene glycol and of an amine catalyst, in a reactor at 150-250° C., in batch mode. The diester monomer then obtained is purified by filtration, ion exchange and/or passage over activated carbon, before being crystallized and recovered by filtration.

Patent JP3715812 describes the production of refined BHET from PET in flake form. The depolymerization step consists of the glycolysis of the PET flakes, which have been pretreated beforehand by washing with water in solid form, in the presence of ethylene glycol and of a catalyst in a stirred reactor at 180° C. to remove the residual water, and then at 195-200° C. The depolymerization is followed by a step of prepurification of the reaction effluent by cooling, filtration, adsorption and treatment on ion-exchange resin, this step being presented as being very important, being carried out before the evaporation of the glycol and the purification of the BHET. The prepurification makes it possible to prevent the repolymerization of the BHET in the subsequent purification steps.

Lastly, patent application FR 3053691 describes a process for depolymerization of a polyester feedstock comprising opaque PET and in particular from 0.1% to 10% by weight of pigments, by glycolysis in the presence of ethylene glycol. A purified BHET effluent is obtained after particular steps of separation and of purification. Said patent application envisages the possibility of reactive extrusion in a first step of conditioning of the feedstock to initiate the depolymerization reaction.

The present invention aims to improve these processes of depolymerization by alcoholysis or glycolysis of a polyester feedstock, and in particular that of the application FR 3053691. More particularly, the present invention aims to improve the phase of conditioning of the polyester feedstock and the mixing thereof with at least one alcohol stream, as depolymerization agent, upstream of the depolymerization step, so as to obtain a homogeneous stream exhibiting a sufficiently low viscosity, in particular of less than or equal to 50 mPa·s, thus enabling a reaction step (namely a depolymerization step) which is optimal in particular in terms of efficiency of the reaction, of required stirring power, and of operating costs.

### SUMMARY OF THE INVENTION

The aim of the invention is thus a process for depolymerization of a polyester feedstock, comprising:
a) a conditioning step using a means for at least partially melting the polyester feedstock and at least one static or dynamic mixer, located downstream of the means for at least partially melting the polyester feedstock, to produce a conditioned feedstock stream,
conditioning step a) being operated at a temperature of between 200 and 300° C. and fed at least with the polyester feedstock and an alcohol stream comprising an alcohol compound, with a ratio by weight of the alcohol stream in relation to the polyester feedstock of between 0.03 and 6.00,
the means for at least partially melting the polyester feedstock being at least fed with the polyester feedstock,
each static or dynamic mixer being fed with at least a fraction of the alcohol stream and with a polyester stream, with a volume degree of dilution with alcohol compound of between 3% and 70%, the volume degree of dilution with alcohol compound being the ratio between the volume flow rate of the fraction of the alcohol stream which feeds the static or dynamic mixer under consideration and the sum of the volume flow rates of the alcohol stream fraction and of the polyester stream which feed the static or dynamic mixer under consideration, the polyester stream which feeds a static or dynamic mixer comprising the polyester feedstock and all of the fractions of the alcohol stream that are introduced in step a) upstream of the static or dynamic mixer under consideration;
b) a step of depolymerization, which is fed at least with the conditioned feedstock stream obtained from step a), and is operated at a temperature of between 150 and 300° C., with a residence time of between 0.1 and 10 h and with a ratio by weight between the total amount of alcohol compound present in step b) and the amount of diester contained in the conditioned feedstock stream of between 0.3 and 8.0.

One advantage of the invention is that of improving the step of conditioning the polyester feedstock, so as to improve the homogenization of the mixture of the polyester feedstock with at least one depolymerization agent, in particular an alcohol stream, and of obtaining, at the outlet of the conditioning section, a homogeneous polyester-depolymerization agent mixture exhibiting a viscosity advantageously of less than or equal to 50 mPa·s, preferably less than or equal to 30 mPa·s and very preferentially less than or equal to 15 mPa·s. Such a mixture thus has the advantage of resulting in a sufficiently low effective viscosity in the reaction section, in order to make it possible to use a reasonable (i.e. limited) stirring power in the reaction section, and in particular in the reactor connected directly to the conditioning unit, which facilitates the operability of the depolymerization process and limits the costs required to implement it. The process according to the invention thus facilitates the dispersion and homogenization of the feedstock with at least one alcohol stream, which makes it possible to improve the efficiency of the depolymerization reaction while reducing the stirring power required for this dispersion and homogenization in the reaction section.

The present invention thus makes it possible to effectively premix the polyester feedstock with at least a portion of the depolymerization agent, in particular of the monoalcohol or of the diol, required for the depolymerization of the polyester, in particular of the PET, while observing the technical constraints imposed by the mixing equipment used, in particular by the stirring system of the reaction section but also by equipment used in the conditioning section, for example static or dynamic mixers for which it is recommended to avoid excessive differences in viscosity between the fluids to be mixed. Typically, static mixers are used to mix fluids having a ratio of viscosity between said fluids which varies by up to 1000 (i.e. ≤1000). However, the present invention makes it possible to effectively mix a polyester feedstock comprising PET, having a viscosity in the molten state which is typically between 300 and 800 Pa·s, with an alcohol stream, in particular a stream of methanol or a stream of ethylene glycol, having a viscosity which varies between 1 and 0.1 mPa·s within the range of temperatures at which the mixing is carried out, that is to say a ratio of viscosity between these two fluids in a range of from approximately 1×10⁵-1×10⁶, which is very high and usually of little compatibility with the technical constraints of the static or dynamic mixers.

Lastly, one advantage of the invention is that it is able to treat any type of polyester wastes, which increasingly comprise pigments, dyes and other polymers, such as azure, coloured, opaque and multilayer PETs.

### LIST OF FIGURES

FIG. 1 represents one particular embodiment of the process according to the invention implementing a depolymerization by glycolysis of the presence in ethylene glycol, and comprising:

a step (a) of conditioning of the polyester feedstock (1) preferably comprising PET, using a means (A) for at least partially melting the polyester feedstock and obtaining an at least partially molten polyester feedstock (1*), and four static mixers (M1), (M2), (M3), (M4) in series, each respectively fed with the fractions (2), (4), (6) and (8) of an ethylene glycol stream (11), and each producing a respective polyester stream (3), (5), (7) and (9), comprising the, at least partially molten, polyester feedstock (1) mixed with the fraction(s) of the ethylene glycol stream that has/have already been introduced;

a depolymerization step (b) fed with the conditioned feedstock (9) obtained from conditioning step a) and with a diol effluent (12); and

a step (c) making it possible to separate a diol stream (10) and a diester monomer stream (13), the diol stream (10) being able to be purified and mixed with an external diol stream (14) before being recycled to the conditioning (a) and depolymerization (b) steps, and a BHET effluent (14).

FIG. 2 represents another particular embodiment of the process according to the invention implementing a depolymerization by glycolysis of the presence in ethylene glycol, and comprising:

a step (a) of conditioning of the polyester feedstock (1) preferably comprising PET, using an extruder (A) fed with the polyester feedstock (1) and a fraction (2) of an ethylene glycol stream (11), producing a mixture (3) and followed by two static mixers (M1), (M2) in series, each respectively fed with the fractions (4) and (6) of the ethylene glycol stream (11), and each producing a respective polyester stream (5) and (7), comprising the, at least partially molten, polyester feedstock (1) mixed with the fraction(s) of the ethylene glycol stream that has/have already been introduced;

a depolymerization step (b) fed with the conditioned feedstock (7) obtained from conditioning step a) and with a diol effluent (12); and

a step (c) making it possible to separate a diol stream (10) and a diester monomer stream (13), the diol stream (10) being able to be purified and mixed with an external diol stream (14) before being recycled to the conditioning (a) and depolymerization (b) steps, and a BHET effluent (14).

### DESCRIPTION OF THE EMBODIMENTS

According to the invention, polyethylene terephthalate or poly(ethylene terephthalate), also simply called PET, has an elementary repeating unit of formula:

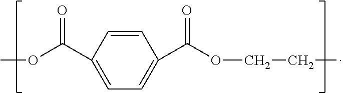

Conventionally, PET is obtained by polycondensation of terephthalic acid (PTA) or dimethyl terephthalate (DMT) with ethylene glycol.

In the continuation of the text, the expression “per mole of diester in said polyester feedstock” corresponds to the number of moles of —[O—CO—O—(C₆H₄)—CO—O—CH₂—CH₂]— unit in the polyester feedstock, which in particular is the diester unit obtained from the reaction of PTA and ethylene glycol.

According to the invention, the term “monomer” or “diester monomer” or else “diester” advantageously denotes a repeating unit of a polyester polymer.

According to a preferred embodiment of the invention, the term “monomer” or “diester monomer” or else “diester” is defined as a diester of a dicarboxylic acid, preferably of a dicarboxylic acid and preferentially of terephthalic acid, and of a diol comprising preferably between 2 and 12 carbon atoms, preferentially between 2 and 4 carbon atoms, the preferred diol being ethylene glycol. According to this embodiment, the term “monomer” or “diester monomer” preferably denotes bis(2-hydroxyethyl) terephthalate (BHET) of chemical formula HOC₂H₄—CO₂—(C₆H₄)—CO₂—C₂H₄OH, in which —(C₆H₄)— represents an aromatic ring, this in particular being the diester unit obtained from the reaction of PTA and ethylene glycol.

According to another embodiment of the invention, the term “monomer” or “diester monomer” may define a diester of a dicarboxylic acid, preferably of a dicarboxylic acid and preferentially of terephthalic acid, and of a monoalcohol comprising preferably between 1 and 10 carbon atoms, preferentially between 1 and 3 carbon atoms, and with preference methanol, ethanol, propanol, or mixtures thereof. According to this embodiment, the term “monomer” or “diester monomer” very preferably denotes dimethyl terephthalate (DMT), of chemical formula CH₃—CO₂—(C₆H₄)—CO₂—CH₃, in which —(C₆H₄)— represents an aromatic ring.

The term “oligomer” typically denotes a polymer of small size, consisting generally of 2 to 20 elementary repeating units, for example between 2 and 5 elementary repeating units. Preferably, the term “ester oligomer” or “BHET oligomer” denotes a terephthalate ester oligomer comprising between 2 and 20, preferably between 2 and 5, elementary repeating units of formula —[O—CO—(C₆H₄)—CO—O—C₂H₄]—, where —(C₆H₄)— is an aromatic ring.

According to the invention, the term “monoalcohol” denotes a compound comprising a single hydroxyl-OH group and preferably comprising between 1 and 10 carbon atoms, preferentially between 1 and 3 carbon atoms. Preferably, the monoalcohol is chosen from methanol, ethanol, propanol and mixtures thereof, the preferred monoalcohol being methanol.

According to the invention, the terms “diol” and “glycol” are used without distinction and correspond to compounds comprising 2 hydroxyl-OH groups and preferably comprising between 2 and 12 carbon atoms, preferentially between 2 and 4 carbon atoms. The preferred diol is ethylene glycol, also referred to as monoethylene glycol or MEG.

According to the invention, the term “alcohol compound” denotes a monoalcohol or a diol, as defined hereinabove. The alcohol compound is advantageously a depolymerization agent required for the depolymerization by alcoholysis or glycolysis of the polyester feedstock. According to a very preferred embodiment, the alcohol compound is a diol comprising between 2 and 12 carbon atoms, preferentially between 2 and 4 carbon atoms, and very preferentially is ethylene glycol. According to another embodiment of the invention, the alcohol compound is a monoalcohol comprising preferably between 1 and 10 carbon atoms, preferentially between 1 and 3 carbon atoms, with preference chosen from methanol, ethanol, propanol and mixtures thereof, the preferred monoalcohol being methanol.

The alcohol stream, used in the steps of the process of the invention, comprises, preferably consists of, the alcohol compound advantageously defined above. The alcohol stream preferentially comprises at least 95% by weight of alcohol compound, and in particular at least 95% by weight of monoalcohol or of diol. Very preferably, the alcohol stream comprises at least 95% by weight of ethylene glycol.

Very preferably, the alcohol compound is ethylene glycol, and the alcohol stream is thus a diol stream and more precisely a stream of ethylene glycol, and the target diester monomer is BHET.

The term “dye” defines a substance that is soluble in the polyester material and that is used to colour it. The dye can be of natural or synthetic origin.

According to the invention, the term “pigment”, more particularly opacifying and/or colouring pigment, defines a finely divided substance which is insoluble in particular in the polyester material. The pigments are in the form of solid particles with a size of generally between 0.1 and 10 μm, and predominantly between 0.4 and 0.8 μm. They are often of inorganic nature. The pigments generally used, notably for opacifying, are metal oxides, such as TiO₂, CoAl₂O₄ or Fe₂O₃, silicates, polysulfides and carbon black.

The terms “upstream” and “downstream” should be understood as a function of the general flow of the stream in the process.

The terms “static or dynamic mixer” and “mixer” are used without distinction and correspond to mixing equipment well known to a person skilled in the art as static mixer or dynamic mixer.

According to the invention, the viscosity is defined as being a dynamic viscosity, in particular measured at a temperature of 250° C. and at a shear rate of 100 s⁻¹ using a viscometer, preferably using a plate-plate viscometer, for example of DHR3 type from TA Instruments.

According to the present invention, the expressions “of between . . . and . . . ” and “between . . . and . . . ” are equivalent and mean that the limit values of the interval are included in the described range of values. If such were not the case and if the limit values were not included in the described range, such a clarification will be given by the present invention.

For the purposes of the present invention, the various ranges of parameters for a given step, such as the pressure ranges and the temperature ranges, may be used alone or in combination. For example, within the meaning of the present invention, a range of preferred pressure values can be combined with a range of more preferred temperature values.

In the text hereinbelow, particular embodiments of the invention may be described. They may be implemented separately or combined together without limitation of combinations when this is technically feasible.

### Feedstock

The process according to the invention is fed with a polyester feedstock comprising at least one polyester, that is to say a polymer, the repeating unit of the main chain of which contains an ester function. The polyester feedstock preferably comprises polyethylene terephthalate (PET), for example clear PET and/or coloured PET and/or opaque PET.

Said polyester feedstock is advantageously a polyester feedstock to be recycled, obtained from waste, in particular plastic waste, collection and sorting channels. Said polyester feedstock may originate, for example, from the collection of bottles, container trays, films, resins and/or fibres consisting of polyethylene terephthalate.

Preferably, the polyester feedstock comprises at least 50% by weight, preferably at least 70% by weight, and with preference at least 90% by weight of polyethylene terephthalate (PET), the maximum being 100% by weight of PET.

Preferably, said polyester feedstock comprises at least one PET chosen from clear, coloured, opaque, dark and multilayer PET, and mixtures thereof. Very particularly, said polyester feedstock comprises at least 10% by weight of opaque PET, very preferably at least 15% by weight of opaque PET, said opaque PET advantageously being opaque PET to be recycled, i.e. obtained from collection and sorting channels. The polyester feedstock may comprise 100% by weight of opaque PET, very preferably less than 70% by weight of opaque PET.

Said polyester feedstock may comprise pigments and/or dyes. For example, the polyester feedstock may comprise from 0.1% to 10% by weight of pigments, in particular from 0.1% to 5% by weight of pigments. It may also comprise in particular from 0.005% to 1% of dyes, preferably from 0.01% to 0.2% by weight of dyes.

In collection and sorting channels, the polyester wastes are washed and ground before constituting the polyester feedstock of the process according to the invention.

The polyester feedstock may be totally or partly in the form of flakes, the greatest length of which is less than 10 cm, preferentially between 5 and 25 mm, or in micronized solid form, i.e. in the form of particles preferably between 10 micrometres (μm) and 1 mm in size. The feedstock may also comprise “macroscopic” impurities, preferably less than 5% by weight, preferentially less than 3% by weight of “macroscopic” impurities, such as glass, metal, plastics other than polyester (for example PP, PEHD, etc.), wood, paper, cardboard or inorganic elements. Said polyester feedstock may also be totally or partly in the form of fibres, such as textile fibres, which have optionally been pretreated to remove cotton or polyamide fibres, or any textile fibre other than polyester, or yet other fibres such as tyre fibres, which have optionally been pretreated notably to remove polyamide fibres or rubber or polybutadiene residues. Said polyester feedstock may also comprise polyester obtained from production rejects of polyester polymerization and/or transformation processes. The polyester feedstock may also comprise elements used as polymerization catalysts and/or stabilizers in PET production processes, such as antimony, titanium or tin.

### Conditioning Step a)

The process according to the invention comprises a conditioning step a) which at least uses a means for at least partially melting the polyester feedstock and at least one static or dynamic mixer located downstream of the means for at least partially melting the polyester feedstock. Conditioning step a) makes it possible to obtain a conditioned feedstock stream.

The assembly comprising, preferably consisting of, the means for at least partially melting the polyester feedstock and the static or dynamic mixer(s) constitutes a section referred to as conditioning section.

Said conditioning section of step a) thus makes it possible on the one hand to heat and pressurize said polyester feedstock to the operating conditions of depolymerization step b), and on the other hand to contact and premix the polyester feedstock with at least a portion of the alcohol compound required for the depolymerization.

Advantageously, conditioning step a) is fed with the polyester feedstock and an alcohol stream such that the ratio by weight of the alcohol stream in relation to the polyester feedstock, that is to say the ratio between the flow rate by weight of the alcohol stream which feeds step a) and the flow rate by weight of the polyester feedstock which feeds step a), is between 0.03 and 6.00, preferably between 0.05 and 5.00, preferentially between 0.10 and 4.00, with preference between 0.50 and 3.00. Very advantageously, the alcohol stream corresponds to at least a fraction of the alcoholic effluent obtained from optional step c). The temperature at which step a) is implemented, in particular in the means for at least partially melting the polyester feedstock and in the static or dynamic mixer(s), is advantageously between 200 and 300° C., preferentially between 250 and 290° C. This temperature is kept as low as possible to minimize the thermal degradation of the polyester, but must be sufficient to at least partially melt the polyester feedstock. Preferably, the conditioning section is operated under inert atmosphere, to limit the introduction of oxygen into the system and hence the oxidation of the polyester feedstock.

Advantageously, the means for at least partially melting the polyester feedstock makes it possible to mix and melt, at least partially, the polyester feedstock, and more particularly to at least partially melt the PET of the polyester feedstock. Preferably, the means for at least partially melting the polyester feedstock is an extruder, in particular a twin-screw or single-screw extruder. Said means is advantageously carried out at a temperature of between 200 and 300° C., preferentially between 250 and 290° C.

The means for at least partially melting the polyester feedstock is advantageously at least fed with the polyester feedstock, for example in flake form, and makes it possible to obtain a viscous liquid stream, typically having a viscosity of between 0.5 and 600 Pa·s, or indeed more particularly between 1.0 and 500 Pa·s. The viscosity is in particular a dynamic viscosity measured at a temperature of 250° C. and at a shear rate of 100 s⁻¹ using a viscometer, preferably using a plate-plate viscometer, for example of DHR3 type from TA Instruments. In the means for at least partially melting the polyester feedstock, such as an extruder, the polyester feedstock is advantageously progressively heated up to a temperature of between 200 and 300° C., preferentially between 250 and 290° C., and in particular close to or even slightly above the melting point of the polyester, for example of the PET, that it contains, so as to become at least partially liquid (i.e. at least partially molten) at the outlet of said means. Very advantageously, at least 70% by weight of the polyester feedstock, preferably at least 80% by weight, preferentially at least 90% by weight, with preference at least 95% by weight of the polyester feedstock is in liquid form on leaving said means, for example the extruder, of step a).

More particularly, the polyester feedstock feeds the means for at least partially melting the polyester feedstock, said means preferably being an extruder. The feeding of the polyester feedstock is carried out advantageously by any method known to a person skilled in the art, for example via a feed hopper, and may be inertized in order to limit the introduction of oxygen into the process. Advantageously, the means for at least partially melting said feedstock, preferably an extruder, makes it possible to bring the polyester feedstock to a temperature of between 200 and 300° C., preferentially between 250 and 290° C., and to a pressure of preferably between atmospheric pressure (i.e. 0.1 MPa) and 20 MPa, preferably between 0.15 MPa and 10 MPa, conditions under which said polyester feedstock is advantageously at least partially molten, and in particular under which the PET possibly included in the polyester feedstock is at least partially molten, preferably fully molten.

According to a preferred embodiment of the invention, the means for at least partially melting the polyester feedstock, preferably the extruder, may also be fed with a fraction of the alcohol stream which feeds step a), which may help to at least partially liquidize the polyester feedstock and hence may contribute to reducing the viscosity of the stream at the outlet of said means, thus contributing to the overall homogenization of the at least partially molten polyester feedstock and of the alcohol compound in particular in conditioning step a) and also depolymerization step b). Another advantage of this embodiment (that is to say the introduction into the melting means of a fraction of the alcohol stream which feeds conditioning step a)) resides in the fact that this implementation may make it possible to reduce the number of static or dynamic mixers required to achieve a viscosity of the [polyester feedstock+alcohol compound] mixture at the end of step a) (corresponding to the conditioned feedstock stream) of less than or equal to 50 mPa·s, preferably less than or equal to 30 mPa·s and very preferentially less than or equal to 15 mPa·s. When a fraction of the alcohol stream which feeds step a) is introduced into the means for at least partially melting the polyester feedstock, the amount of alcohol compound which feeds said means is preferably adjusted such that the ratio by weight between said fraction of the alcohol stream which feeds said means and the polyester feedstock which feeds said means is between 0.001 and 0.100, preferably between 0.003 and 0.050, very preferably between 0.005 and 0.030.

Preferably, the residence time in the means for at least partially melting the polyester feedstock is advantageously less than or equal to 5 min, preferably less than or equal to 2 min, and with preference greater than 1 second, preferentially greater than or equal to 10 seconds. Said residence time is defined as the volume available in said means divided by the volume flow rate of the polyester feedstock.

The means for at least partially melting the polyester feedstock may advantageously be connected to a vacuum extraction system so as to remove impurities, such as dissolved gases, light organic compounds and/or moisture present in the feedstock.

The means for at least partially melting the polyester feedstock, preferably an extruder, may also advantageously comprise at the outlet a filtration system thus making it possible to remove solid particles having a size of greater than 20 μm and preferably of less than 2 cm, such as sand, wood, or metal particles.

According to a particular embodiment, the means for at least partially melting the polyester feedstock, preferably an extruder, is directly connected, at the outlet, to a first filtration system, in particular a filter, designed to remove solid particles having a size typically of greater than or equal to 1000 μm, preferably greater than or equal to 500 μm, preferably greater than or equal to 400 μm, preferentially greater than or equal to 300 μm, followed by a melt pump or a gear pump making it possible to maintain and/or increase the pressure, followed by a second filtration system designed to remove solid particles having a size typically of greater than or equal to 60 μm, preferably greater than or equal to 20 μm. Thus, in this particular embodiment, the conditioning section comprises:
the means for at least partially melting the polyester feedstock, preferably an extruder, making it possible to obtain an at least partially molten polyester feedstock, preferably at a pressure of typically between 0.1 MPa and 15.0 MPa, with preference between 0.15 MPa and 1.5 MPa, then
a first filtration system, in particular a filter, designed to remove, from the at least partially molten feedstock obtained from said means, solid particles having a size typically of greater than or equal to 1000 μm, preferably greater than or equal to 500 μm, preferably greater than or equal to 400 μm, preferentially greater than or equal to 300 μm, then
a melt pump or a gear pump, which in particular makes it possible to maintain the pressure and/or increase the pressure in the conditioning section at/to a pressure of greater than or equal to the pressure at the outlet of the means for at least partially melting the polyester feedstock, preferably at/to a pressure of between 0.1 MPa and 15.0 MPa, preferentially between 1 MPa and 15.0 MPa, with preference between 1 MPa and 7.0 MPa, then
a second filtration system, in particular a filter, designed to remove solid particles having a size typically of greater than or equal to 60 μm, preferably greater than or equal to 20 μm, then
at least one static or dynamic mixer, as advantageously described hereinafter.

According to another particular embodiment, a system for separation of metals may be installed upstream of the means for at least partially melting the polyester feedstock, so as to remove any metallic impurities in the polyester feedstock.

Advantageously, conditioning step a) uses a means for at least partially melting the polyester feedstock, preferably an extruder, and at least one, preferably between one and five, with preference between two and five, very preferably between two and four, static or dynamic, preferentially static, mixers. The static or dynamic mixer(s) is/are advantageously located downstream of the means for at least partially melting the polyester feedstock. When the conditioning section comprises two or more static or dynamic mixers, the static or dynamic mixers are advantageously in series with respect to one another. Preferably, conditioning step a) uses an extruder, preferably operated at a temperature of between 200 and 300° C., preferentially between 250 and 290° C., and between two and five, preferably between two and four, static or dynamic mixers, operating in series and preferably implemented at a temperature of between 200 and 300° C., preferentially between 250 and 290° C.

Advantageously, each static or dynamic mixer is fed with at least a fraction of the alcohol stream which feeds step a), and with a polyester stream such that, in each mixer, the volume degree of dilution with alcohol compound is between 3% and 70%. The volume degree of dilution with alcohol compound in a static or dynamic mixer corresponds, according to the invention, to the ratio between the volume flow rate of the fraction of the alcohol stream which directly feeds the static or dynamic mixer under consideration and the sum of the volume flow rates of the fraction of the alcohol stream and of the polyester stream which feed the static or dynamic mixer under consideration. For each static or dynamic mixer, the polyester stream corresponds to a stream comprising, preferably consisting of, the, advantageously at least partially molten, polyester feedstock and all of the fractions of the alcohol stream that have been introduced in step a) upstream of the static or dynamic mixer under consideration. In other words, the polyester stream, which feeds a static or dynamic mixer, corresponds to a stream of material comprising, preferably consisting of, the, advantageously at least partially molten, polyester feedstock supplemented with all of the fractions of the alcohol stream that have been introduced into the static or dynamic mixer(s) located upstream of the static or dynamic mixer under consideration and possibly in the means for at least partially melting the polyester feedstock. For example, when the static or dynamic mixer under consideration is the first static or dynamic mixer of the conditioning section and the means for at least partially melting the polyester feedstock is not fed with alcohol compound, the polyester stream then corresponds to the, advantageously at least partially molten, polyester feedstock.

Preferably, the volume degree of dilution with alcohol compound in each static or dynamic mixer is:
between 3% and 50%, preferably between 10% and 35%, and very preferably between 15% and 30%, when the ratio of the viscosities between the polyester stream and the fraction of the alcohol stream which feed the static or dynamic mixer under consideration is greater than or equal to 3500, preferably greater than or equal to 3000,
between 10% and 70%, preferably between 20% and 65%, very preferably between 30% and 65%, or even between 35% and 65%, when the ratio of the viscosities between the polyester stream and the fraction of the alcohol stream which feed the static or dynamic mixer under consideration is less than 3500, preferably less than 3000.

Preferably, the alcohol stream which feeds conditioning step a) is divided into n partial streams of alcohol compound (i.e. into n fractions of the alcohol stream), n being an integer equal to m or m+1, m being an integer equal to the number of static or dynamic mixers used in conditioning step a), each static or dynamic mixer being fed with one of the partial streams of alcohol compound (i.e. with one of the fractions of the alcohol stream which feeds conditioning step a)) such that, in each static or dynamic mixer, the volume degree of dilution with alcohol compound is between 3% and 70%, and preferably:
between 3% and 50%, preferably between 10% and 35%, and very preferably between 15% and 30%, when the ratio of the viscosities between the polyester stream and the fraction of the alcohol stream which feed the static or dynamic mixer under consideration is greater than or equal to 3500, preferably greater than or equal to 3000; or
between 10% and 70%, preferably between 20% and 65%, very preferably between 30% and 65%, or even between 35% and 65%, when the ratio of the viscosities between the polyester stream and the fraction of the alcohol stream which feed the static or dynamic mixer under consideration is less than 3500, preferably less than 3000.

Optionally, a partial stream of alcohol compound (i.e. a fraction of the alcohol stream) may also feed the melting means.

Advantageously, each static or dynamic mixer is operated at a temperature of between 200 and 300° C., preferentially between 250 and 290° C., preferably with a residence time of between 0.5 second and 20 minutes, preferably between 1 second and 5 minutes, with preference between 3 seconds and 1 minute, the residence time being defined here as the ratio between the volume of liquid in the static or dynamic mixer in relation to the sum of the volume flow rates of the polyester stream and of the fraction of the alcohol stream which feed the static or dynamic mixer under consideration.

The alcohol stream which feeds conditioning step a) may advantageously be heated, preferably to a temperature of between 200 and 300° C., preferentially between 250 and 290° C., prior to being introduced into step a), in particular prior to being introduced into the means for at least partially melting the polyester feedstock and/or into the static or dynamic mixer(s), in order to aid in bringing the polyester feedstock to temperature.

According to a preferred embodiment of the invention, conditioning step a) uses an extruder, optionally a filtration system at the extruder outlet, and then two, three or four static or dynamic mixers, operating in series with respect to one another. In this preferred embodiment, the extruder is fed with the polyester feedstock and preferably with a fraction of the alcohol stream, such that the ratio by weight between said fraction of the alcohol stream, which feeds the extruder, and the polyester feedstock which feeds the extruder is between 0.001 and 0.100, preferably between 0.003 and 0.050, with preference between 0.005 and 0.030. The other fraction of the alcohol stream is then divided respectively into two, three or four partial streams of alcohol compound, the number of partial streams of alcohol compound being equal to the number of static or dynamic mixers used, each of the static or dynamic mixers being fed with a polyester stream and one of the partial streams of alcohol compound such that, in each static or dynamic mixer, the volume degree of dilution with alcohol compound is between 3% and 70%, and:
i) with preference between 3% and 50%, preferably between 10% and 35%, and very preferably between 15% and 30%, when the ratio of the viscosities between the polyester stream and the partial stream of alcohol compound which feed the static or dynamic mixer under consideration is greater than or equal to 3500, preferably greater than or equal to 3000; or
ii) with preference between 10% and 70%, preferably between 20% and 65%, very preferably between 30% and 65%, or even between 35% and 65%, when the ratio of the viscosities between the polyester stream and the partial stream of alcohol compound which feed the static or dynamic mixer under consideration is less than 3500, preferably less than 3000.

Preferably, the residence time in the extruder, defined as the volume available in said extruder divided by the volume flow rate of feedstock, is between 0.5 second and one hour, preferably between 0.5 second and 5 minutes, preferably between 1 second and 2 minutes, or between 10 seconds and 2 minutes.

On conclusion of conditioning step a), a conditioned feedstock stream is advantageously obtained. Very advantageously, the conditioned feedstock stream is in liquid form and preferably exhibits a viscosity of less than or equal to 50 mPa·s, preferably less than or equal to 30 mPa·s and very preferentially less than or equal to 15 mPa·s.

### Depolymerization Step b)

The process according to the invention comprises a depolymerization step b). More particularly, the depolymerization of the polyester feedstock, in particular of the PET that it comprises, is implemented by glycolysis when the alcohol compound is a diol, or by alcoholysis when the alcohol compound is a monoalcohol.

Depolymerization step b) is fed at least with the conditioned feedstock stream obtained from conditioning step a) and optionally with a supply of alcohol compound such that the ratio by weight between the total amount of alcohol compound present in step b), corresponding to the sum of the amounts by weight of alcohol compound introduced in step a) and optionally in step b), and the amount by weight of diester contained in the conditioned feedstock stream (i.e. contained in the polyester feedstock and according to a particular embodiment the amount by weight of PET contained in the polyester feedstock), is between 0.3 and 8.0, preferably between 1.0 and 7.0, with preference between 1.5 and 6.0. In other words, depolymerization step b) is fed with the conditioned feedstock stream obtained from conditioning step a) and optionally with a supply of alcohol compound, such that the molar ratio between the total molar amount of alcohol compound introduced in step a) and optionally in step b) in relation to the total molar amount of diester contained in the conditioned feedstock stream (i.e. contained in the polyester feedstock) is respectively between 0.9 and 24.0, preferably between 3.0 and 21.0, with preference between 4.5 and 18.0.

Preferably, depolymerization step b) is fed with the conditioned feedstock stream obtained from step a) and with a supply of alcohol compound, very preferably a supply of methanol or of ethylene glycol, such that the ratio by weight between the total amount by weight of alcohol compound introduced in steps a) and b) in relation to the total amount by weight of diester contained in the conditioned feedstock stream (i.e. contained in the polyester feedstock and according to a particular embodiment the amount of PET contained in the polyester feedstock) is between 0.3 and 8.0, preferably between 1.0 and 7.0, with preference between 1.5 and 6.0 (i.e. a molar ratio of alcohol compound in relation to the diester respectively of between 0.9 and 24.0, preferably approximately between 3.0 and 21.0, with preference between 4.5 and 18.0).

Advantageously, said depolymerization step b) uses one or more reaction sections, preferably at least two reaction sections, with preference between two and four reaction sections, preferably functioning in series. Each reaction section may comprise a reactor, more particularly any type of reactor known to a person skilled in the art which makes it possible to perform a depolymerization or transesterification reaction, and preferably a reactor stirred with a mechanical stirring system and/or with a recirculation loop and/or by fluidization. In each reaction section, the reactor may optionally comprise a conical base which makes it possible to bleed off the impurities. With preference, the depolymerization step b) is performed at least two reaction sections, preferably between two and four reaction sections, functioning in series, the reaction section(s), starting from the second reaction section, being operated at a mutually identical or different temperature which is preferably lower than or equal to the temperature of the first reaction section, preferably lower, and preferentially 10 to 50° C. lower, or even 20 to 40° C. lower, relative to the temperature of the first reaction section.

Depolymerization step b) is operated at a temperature of between 150 and 300° C., preferably between 180 and 290° C., with preference between 210 and 270° C., in particular in the liquid phase. Advantageously, step b) is implemented with a residence time in each reaction section of between 0.1 and 10 h, preferably between 0.25 and 8 h, between 0.5 and 6 h. The residence time in a reaction section is defined as the ratio of the liquid volume of said reaction section to the volume flow rate of the stream exiting said reaction section.

The operating pressure of the reaction section(s) of step b) is determined so as to keep the reaction system in the liquid phase. This pressure is advantageously at least 0.1 MPa, preferentially at least 0.4 MPa, and preferably less than 10 MPa, preferentially less than 5 MPa. The term “reaction system” means all of the constituents and phases present in said step b).

The depolymerization reaction may be carried out in the presence or absence of a catalyst. When the depolymerization reaction is carried out in the presence of a catalyst, the latter can be homogeneous or heterogeneous and chosen from the esterification catalysts known to a person skilled in the art, such as complexes, oxides and salts of antimony, tin or titanium, alkoxides of metals from Groups (I) and (IV) of the Periodic Table of the Elements, organic peroxides, acidic/basic metal oxides, and compounds based on manganese, on zinc, on titanium, on lithium, on magnesium, on calcium or on cobalt.

A preferred heterogeneous catalyst advantageously comprises at least 50% by mass relative to the total mass of the catalyst, preferentially at least 70% by mass, advantageously at least 80% by mass, very advantageously at least 90% by mass and even more advantageously at least 95% by mass of a solid solution consisting of at least one spinel of formula ZₓAl₂O₍₃₊ₓ₎ in which x is between 0 (limit excluded) and 1, and Z is chosen from Co, Fe, Mg, Mn, Ti and Zn and comprising no more than 50% by mass of alumina and of oxide of the element Z. Said preferred heterogeneous catalyst advantageously contains no more than 10% by mass of dopants chosen from silicon, phosphorus and boron, taken alone or as a mixture. For example, and in a non-limiting manner, said solid solution may consist of a mixture of spinel ZnAl₂O₄ and of spinel CoAl₂O₄, or else may consist of a mixture of spinel ZnAl₂O₄, of spinel MgAl₂O₄ and of spinel FeAl₂O₄, or else may consist solely of spinel ZnAl₂O₄.

According to a particular embodiment of the invention, a homogeneous catalyst, preferably chosen from amines, preferably tertiary mono- and diamines, such as for example tetramethylethylenediamine (TMEDA), pentamethyldiethylenetriamine (PMDETA), trimethyltriazacyclononane (TACN), triethylamine (TEA), 4-(N,N-dimethylamino)pyridine (DMAP), 1,4-diazabicyclo[2.2.2]octane (DABCO), N-methylimidazole (NMI), and alkali metal or alkaline earth metal hydroxides, such as for example Mg(OH)₂ and NaOH, may be added in depolymerization step b).

Said depolymerization step is preferably carried out without addition of external catalyst to the polyester feedstock.

Said depolymerization step may advantageously be carried out in the presence of a solid adsorbing agent in powder or shaped form, so as to capture at least a portion of the coloured impurities, thus relieving the strain on any possible purification steps. Said solid adsorbing agent is advantageously an activated carbon.

The depolymerization reaction makes it possible to convert the polyester feedstock into monomers and/or oligomers. Preferably, the depolymerization step makes it possible to convert the polyester of the polyester feedstock, preferably the PET of the polyester feedstock, and possibly its oligomers, into at least one diester monomer, preferably bis(2-hydroxyethyl) terephthalate (BHET) or dimethyl terephthalate (DMT), and possibly oligomers. The conversion of the polyester, preferably of the PET, of the polyester feedstock, on conclusion of depolymerization step b) is greater than 50%, preferably greater than 70%, with preference greater than 85%. Preferably, the molar yield of diester monomer, very preferably of BHET, is greater than 50%, preferably greater than 70%, with preference greater than 85%. The molar yield of diester monomer corresponds to the molar flow rate of diester monomer at the outlet of step b) (i.e. in the reaction effluent) in relation to the number of moles of diester in the polyester feedstock feeding step a).

In parallel, the depolymerization reaction also typically generates a diol, in particular ethylene glycol.

An internal recirculation loop may advantageously be performed in step b), with withdrawal of a fraction of the reaction system, the filtration of this fraction and the reinjection of said filtered fraction into said step b). This internal loop makes it possible to remove the “macroscopic” solid impurities that may be present in the reaction liquid.

Advantageously, depolymerization step b) makes it possible to obtain a reaction effluent, advantageously in essentially liquid form, which comprises the target diester monomer, very preferably the BHET. The reaction effluent may be sent to purification steps to separate the diester monomer, very preferably the BHET, from the other compounds present in the reaction effluent, such as unreacted alcohol compound, the diol generated during the depolymerization, preferably the ethylene glycol generated, impurities such as pigments and/or dyes, or else byproducts that may be generated such as diol dimers or trimers and derivatives thereof (for example esters of diol dimer), in order to obtain a purified diester monomer effluent. In particular, the reaction effluent may be sent to an optional separation step c) to recover an alcoholic effluent preferably essentially composed of the alcohol compound.

### Optional Separation Step c)

The process according to the invention may comprise a separation step c), fed at least with the reaction effluent obtained from step b), and producing at least an alcoholic effluent and a diester monomer effluent.

The main role of optional step c) is to recover all or some of the unreacted alcohol compound, which may then advantageously be recycled to steps a) and/or b). Optional step c) may also make it possible to recover all or some of the diol generated during the depolymerization.

Optional step c) is advantageously implemented in a gas-liquid separation section or a succession of gas-liquid separation sections, advantageously from two to five successive gas-liquid separation sections. Each of the gas-liquid separation sections produces a liquid phase and a gas phase. The liquid phase from the preceding gas-liquid separation section feeds the subsequent gas-liquid separation section. All of the gas phases are recovered to constitute the alcoholic effluent. The liquid phase obtained from the final gas-liquid separation section constitutes the diester monomer effluent.

Advantageously, at least one of the gas-liquid separation sections may be implemented in a falling-film evaporator or a thin-film evaporator. Optional step c) may also implement at least one short-path distillation separation section.

Advantageously, step c) is operated such that the temperature of the liquid phases is kept above a lower temperature value below which the diester monomer, preferably the BHET monomer, precipitates, and below an upper temperature value, above which the diester monomer undergoes significant repolymerization. The temperature in step c) is advantageously between 60 and 250° C., preferably between 90 and 220° C., with preference between 100 and 210° C. Operation as a succession of two to five successive gas-liquid separations is particularly advantageous since it makes it possible to adjust, within each separation, the temperature of the liquid phase in accordance with the above-mentioned constraints.

The pressure in optional step c) is preferably lower than that of step b), so as to vaporize a fraction of the reaction effluent obtained from step b). The pressure in optional step c) is thus advantageously adjusted to allow the evaporation of the diol at a given temperature in each separation section, while minimizing the repolymerization of the monomer and enabling optimum integration in terms of energy. It is preferably between 0.00001 and 0.2 MPa, preferentially between 0.00004 and 0.15 MPa, with preference between 0.00004 and 0.1 MPa.

The gas-liquid separation section(s) is/are advantageously stirred by any method known to a person skilled in the art.

The alcoholic effluent obtained on conclusion of optional step c) comprises unreacted alcohol compound. It may also contain diol, preferably ethylene glycol, generated during the depolymerization and possibly other compounds such as dyes, light alcohols, water, or diethylene glycol. At least a fraction of the alcoholic effluent may advantageously be recycled, preferably after purification and with preference in liquid form (i.e. after condensation), to step a) and/or step b), optionally as a mixture with a supplement of alcohol compound external to the process according to the invention.

All or some of said alcoholic effluent may be treated in a purification step prior to being recycled, preferably in liquid form, to steps a) and/or b). This purification step may comprise, in a non-exhaustive manner, adsorption onto solid (for example onto activated carbon), in order to remove the dyes, and one or more distillations, in order to separate out the impurities, such as diethylene glycol, water and other alcohols.

The diester monomer effluent obtained on conclusion of optional step c) may be transferred to one or more purification steps, so as to obtain a decolourized and purified diester monomer effluent, very preferably a decolourized and purified BHET effluent, which is then able to be polymerized.

According to a particular embodiment, the depolymerization process according to the invention may be integrated into the process described in patent application FR 3053691. In this embodiment, the process according to the invention comprises the optional step c) of separation of the diol and replaces the steps a) of conditioning, b) of depolymerization, and c) of separation of the diol, of the process described in patent application FR 3053691. Thus, in this embodiment, the overall process comprises the depolymerization process according to the invention with the steps a) of conditioning and b) depolymerization and the optional step c) as described above, followed by a step d) of separation of the monomer and by a step e) of purification in particular by decolourization, such as those described in the application FR 3053691.

The process according to the invention thus makes it possible to obtain, starting from any type of polyester waste, for example comprising opaque PET, an effluent comprising a diester monomer, in a manner which is optimized both in respect of operability of the process and operating costs. Said diester monomer obtained may then, preferably after purification, be polymerized, in the presence or absence of ethylene glycol, terephthalic acid and/or dimethyl terephthalate, to produce PET which is visually indistinguishable from virgin PET.

The following figures and examples illustrate the invention without limiting the scope thereof.

### EXAMPLES

In the examples which follow, only conditioning steps a) are described precisely.

### Example 1 (in Accordance)

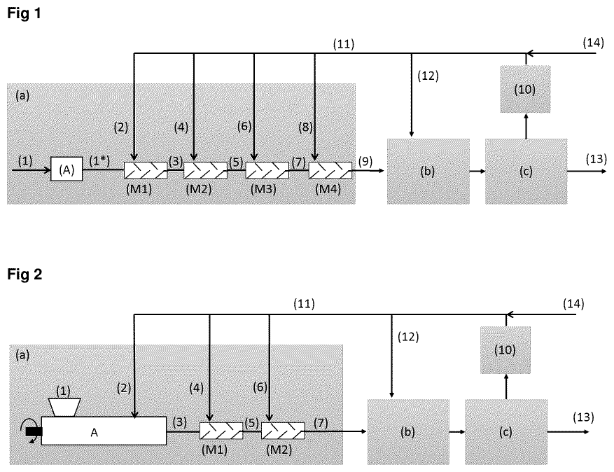

In this example, the depolymerization process corresponds to the embodiment illustrated schematically in FIG. 1, in which the conditioning section comprises:
an extruder A, which comprises a feed hopper through which the extruder is fed with PET feedstock 1 obtained from the collection and sorting channel, at a flow rate of 50 kg/h; followed by
four static mixers M1, M2, M3, M4, in series.

The PET feedstock is in flake form and comprises: 95.72% by weight of PET; 1.24% by weight of pigments; 0.04% by weight of dyes; and 3.00% by weight of paper, wood, metal, sand, etc. impurities.

Each mixer M1, M2, M3, M4 is fed with a respective PET stream 1, 3, 5 and 7 and with a respective fraction 2, 4, 6, 8 of the ethylene glycol stream 11 obtained from step c) of separation of the diol (ethylene glycol or MEG).

The conditioning section is implemented at a temperature of 250° C. and at a pressure of 1.0 MPa (10 bar).

Table 1 presents both the amounts of ethylene glycol (MEG) introduced into each mixer and the change in the viscosities of the PET streams at the inlet/outlet of each static mixer under the temperature and pressure operating conditions. Table 1 also indicates the ratio of the viscosities between the PET stream and the MEG stream which enter each static mixer. The volume degree of dilution with MEG in each mixer corresponds:
for the mixer M1, to the degree of dilution with MEG given for stream 3;
for the mixer M2, to the degree of dilution with MEG given for stream 5;
for the mixer M3, to the degree of dilution with MEG given for stream 7;
for the mixer M4, to the degree of dilution with MEG given for stream 9.

TABLE 1

| | | | | | | Ratio |
|---|---|---|---|---|---|---|
| | | | Degree of | | | of the |
| | | | dilution | | Viscosity of | viscosities |
| | | | with | Ratio by | the stream | between the |
| | Nature | Flow | MEG | weight of | under | PET and |
| | of the | rate | (vol %) | MEG_{cumulative}/PET | consideration | MEG |
| Stream | stream | (kg/h) | (mixer) | (kg/kg) | (mPa · s) | streams |
| 1 | PET | 50.0 | — | — | 500 000 | 877 193 |
| | feedstock | | | | | |
| 2 | MEG | 10.0 | — | — | 0.57 | |
| 3 | PET | 60.0 | 20% (M1) | 0.20 | 33 283 | 58 391 |
| | stream | | | | | |
| 4 | MEG | 17.0 | — | — | 0.57 | |
| 5 | PET | 77.0 | 25% (M2) | 0.54 | 2098 | 3681 |
| | stream | | | | | |
| 6 | MEG | 29.0 | — | — | 0.57 | |
| 7 | PET | 106.0 | 30% (M3) | 1.12 | 17.89 | 31 |
| | stream | | | | | |
| 8 | MEG | 49.0 | — | — | 0.57 | |
| 9 | PET | 155.0 | 34% (M4) | 2.00 | 1.5 | — |
| | stream | | | | | |

On conclusion of conditioning step a) implementing an extrusion followed by four static mixers and in which MEG is progressively introduced up to a ratio by weight of 2 in relation to the PET feedstock (2 parts of MEG for 1 part of PET feedstock), the viscosity of the conditioned feedstock stream is 1.5 mPa·s (i.e. less than 15 mPa·s), this being achieved while observing the technical constraints imposed by the static mixers in relation to the viscosities of the streams involved. Such a viscosity then facilitates the homogenization of the mixture in the reaction section which follows mixer M4.

### Example 2 (in Accordance)

In this example, the depolymerization process corresponds to the embodiment illustrated schematically in FIG. 2, in which the conditioning section comprises:
an extruder A, which comprises a feed hopper through which the extruder is fed with PET feedstock 1 obtained from the collection and sorting channel, at a flow rate of 50 kg/h; then
two static mixers M1 and M2, in series.

The PET feedstock is the same as that of Example 1: it is in flake form and comprises: 95.72% by weight of PET; 1.24% by weight of pigments; 0.04% by weight of dyes; and 3.00% by weight of paper, wood, metal, sand, etc. impurities.

The extruder is fed with a fraction 2 of the ethylene glycol stream 11 obtained from step c) of separation of the diol (ethylene glycol or MEG).

Each mixer M1 and M2 is fed with a respective PET stream 3 and 5 and with a respective fraction 4 and 6 of the ethylene glycol stream 11 obtained from step c) of separation of the diol (ethylene glycol or MEG).

The conditioning section is implemented at a temperature of 250° C. and at a pressure of 1.0 MPa (10 bar).

Table 2 presents both the amounts of ethylene glycol (MEG) introduced into each mixer and the change in the viscosities of the PET streams at the inlet/outlet of each static mixer under the temperature and pressure operating conditions. Table 2 also indicates the ratio of the viscosities between the PET stream and the MEG stream which enter the extruder and each static mixer. The volume degree of dilution with MEG in each mixer and in the extruder corresponds:
for the extruder A, to the degree of dilution with MEG given for stream 3;
for the mixer M1, to the degree of dilution with MEG given for stream 5;
for the mixer M2, to the degree of dilution with MEG given for stream 7.

TABLE 2

| | | | | | | Ratio |
|---|---|---|---|---|---|---|
| | | | Degree of | | Viscosity | of the |
| | | | dilution | | of the | viscosities |
| | | | with | Ratio by | stream | between the |
| | Nature | Flow | MEG (vol %) | weight of | under | PET and |
| | of the | rate | (extruder/ | MEG_{cumulative}/PET | consideration | MEG |
| Stream | stream | (kg/h) | mixer) | (kg/kg) | (mPa · s) | streams |
| 1 | PET | 50.0 | — | — | 500 000 | 877 193 |
| | feedstock | | | | | |
| 2 | MEG | 0.41 | — | — | 0.57 | |
| 3 | PET | 50.41 | 1% (A) | 0.01 | 20 000 | 35 088 |
| | stream | | | | | |
| 4 | MEG | 17.5 | — | — | 0.57 | |
| 5 | PET | 67.91 | 30% (M1) | 0.36 | 7536 | 13 221 |
| | stream | | | | | |
| 6 | MEG | 82.09 | — | — | 0.57 | |
| 7 | PET | 150.0 | 58% (M2) | 2.00 | 8.8 | — |
| | stream | | | | | |

On conclusion of conditioning step a) implementing a reactive extrusion followed by two static mixers and in which MEG is progressively introduced up to a ratio by weight of 2 in relation to the PET feedstock (2 parts of MEG for 1 part of PET feedstock), the viscosity of the conditioned feedstock stream is less than 10 mPa·s (8.8 mPa·s), this being achieved while observing the technical constraints imposed by the static mixers in relation to the viscosities of the streams involved. Such a viscosity then facilitates the homogenization of the mixture in the reaction section which follows mixer M2.

1. A process for depolymerization of a polyester feedstock, comprising:
a) a conditioning step using a means for at least partially melting the polyester feedstock and at least one static or dynamic mixer, located downstream of the means for at least partially melting the polyester feedstock, to produce a conditioned feedstock stream,
conditioning step a) being operated at a temperature of between 200 and 300° C. and fed at least with the polyester feedstock and an alcohol stream comprising an alcohol compound, with a ratio by weight of the alcohol stream in relation to the polyester feedstock of between 0.03 and 6.00,
the means for at least partially melting the polyester feedstock being at least fed with the polyester feedstock,
each static or dynamic mixer being fed with at least a fraction of the alcohol stream and with a polyester stream, with a volume degree of dilution with alcohol compound of between 3% and 70%, the volume degree of dilution with alcohol compound being the ratio between the volume flow rate of the alcohol stream fraction which feeds the static or dynamic mixer under consideration and the sum of the volume flow rates of the alcohol stream fraction and of the polyester stream which feed the static or dynamic mixer under consideration, the polyester stream which feeds a static or dynamic mixer comprising the polyester feedstock and all of the fractions of the alcohol stream that are introduced in step a) upstream of the static or dynamic mixer under consideration;
b) a step of depolymerization, which is fed at least with the conditioned feedstock stream obtained from step a), and is operated at a temperature of between 150 and 300° C., with a residence time of between 0.1 and 10 h and with a ratio by weight between the total amount of alcohol compound present in step b) and the amount of diester contained in the conditioned feedstock stream of between 0.3 and 8.0.

2. The process according to claim 1, wherein, in step a), the ratio by weight of the alcohol stream in relation to the polyester feedstock is between 0.05 and 5.00, preferentially between 0.10 and 4.00, with preference between 0.50 and 3.00.

3. The process according to claim 1, wherein the alcohol compound is a monoalcohol, preferably chosen from methanol, ethanol, propanol and mixtures thereof, preferentially methanol, or a diol such as ethylene glycol.

4. The process according to claim 1, wherein conditioning step a) uses between one and five static or dynamic mixers, preferably between two and five static or dynamic mixers, with preference between two and four static or dynamic mixers, said static or dynamic mixers being in series with respect to one another.

5. The process according to claim 1, wherein, in each static or dynamic mixer used in step a), the volume degree of dilution with alcohol compound is:
between 3% and 50%, preferably between 10% and 35%, and very preferably between 15% and 30%, when the ratio of the viscosities between the polyester stream and the fraction of the alcohol stream which feed the static or dynamic mixer under consideration is greater than or equal to 3500, preferably greater than or equal to 3000,
between 10% and 70%, preferably between 20% and 65%, very preferably between 30% and 65%, or even between 35% and 65%, when the ratio of the viscosities between the polyester stream and the fraction of the alcohol stream which feed the static or dynamic mixer under consideration is less than 3500, preferably less than 3000.

6. The process according to claim 1, wherein conditioning step a) is operated at a temperature of between 250 and 290° C.

7. The process according to claim 1, wherein the means for at least partially melting the polyester feedstock, preferably an extruder, is also fed with a fraction of the alcohol stream which feeds step a), preferably in a ratio by weight between said fraction of the alcohol stream which feeds said means and the polyester feedstock which feeds said means of between 0.001 and 0.100, preferably between 0.003 and 0.050, very preferably between 0.005 and 0.030.

8. The process according to claim 1, wherein the means for at least partially melting the polyester feedstock, preferably an extruder, is operated at a temperature of between 200 and 300° C., preferentially between 250 and 290° C.

9. The process according to claim 1, wherein each static or dynamic mixer is operated at a temperature of between 200 and 300° C., preferentially between 250 and 290° C., preferably with a residence time of between 0.5 second and 20 minutes, preferably between 1 second and 5 minutes, with preference between 3 seconds and 1 minute, the residence time being defined as the ratio between the volume of liquid in the static or dynamic mixer in relation to the sum of the volume flow rates of the polyester stream and of the fraction of the alcohol stream which feed the static or dynamic mixer under consideration.

10. The process according to claim 1, wherein conditioning step a) uses an extruder, followed by two, three or four static or dynamic mixers, placed in series, the extruder being fed with the polyester feedstock and with a fraction of the alcohol stream, such that the ratio by weight between said fraction of the alcohol stream, which feeds the extruder, and the polyester feedstock which feeds the extruder is between 0.001 and 0.100, preferably between 0.003 and 0.050, very preferably between 0.005 and 0.030, the other fraction of the alcohol stream being divided into two, three or four partial streams of alcohol compound, the number of partial streams of alcohol compound being equal to the number of static or dynamic mixers used, each of the static or dynamic mixers being fed with a polyester stream and one of the partial streams of alcohol compound such that, in each static or dynamic mixer, the volume degree of dilution with alcohol compound is:
between 3% and 50%, preferably between 10% and 35%, and very preferably between 15% and 30%, when the ratio of the viscosities between the polyester stream and the partial stream of alcohol compound which feed the static or dynamic mixer under consideration is greater than or equal to 3500, preferably greater than or equal to 3000, or
between 10% and 70%, preferably between 20% and 65%, very preferably between 30% and 65%, or even between 35% and 65%, when the ratio of the viscosities between the polyester stream and the partial stream of alcohol compound which feed the static or dynamic mixer under consideration is less than 3500, preferably less than 3000.

11. The process according to claim 1, wherein the ratio by weight between the total amount of alcohol compound present in step b) and the amount of diester contained in the conditioned feedstock stream is between 1.0 and 7.0, preferably between 1.5 and 6.0.

12. The process according to claim 1, wherein depolymerization step b) is operated at a temperature of between 180 and 290° C., preferably between 210 and 270° C.

13. The process according to claim 1, comprising a separation step c) for producing an alcoholic effluent and a diester monomer effluent, and wherein:
step c) is fed at least with a reaction effluent obtained from step b),
step c) is operated at a temperature of between 60 and 250° C., at a pressure lower than that of step b) and
step c) uses one to five successive gas-liquid separation sections, preferably two to five successive gas-liquid separation sections, each gas-liquid separation section producing a liquid phase and a gas phase, the liquid phase from the preceding gas-liquid separation section feeding the subsequent gas-liquid separation section, the liquid phase obtained from the final gas-liquid separation section constituting the diester monomer effluent, all of the gas phases being recovered to at least partially constitute the alcoholic effluent.

14. The process according to claim 13, wherein the alcohol stream which feeds step a) is at least a fraction of the alcoholic effluent obtained from step c).

15. The process according to claim 1, wherein the polyester feedstock comprises polyethylene terephthalate, advantageously at least 50% by weight, preferably at least 70% by weight, with preference at least 90% by weight of polyethylene terephthalate.

16. The process according to claim 1, wherein, in each static or dynamic mixer used in step a), the volume degree of dilution with alcohol compound is:
between 10% and 35%, when the ratio of the viscosities between the polyester stream and the fraction of the alcohol stream which feed the static or dynamic mixer under consideration is greater than or equal to 3000,
between 20% and 65%, when the ratio of the viscosities between the polyester stream and the fraction of the alcohol stream which feed the static or dynamic mixer under consideration is less than 3000.

17. The process according to claim 1, wherein the means for at least partially melting the polyester feedstock is an extruder, and the extruder is also fed with a fraction of the alcohol stream which feeds step a), in a ratio by weight between said fraction of the alcohol stream which feeds said means and the polyester feedstock which feeds said means of between 0.003 and 0.050.

18. The process according to claim 1, wherein the means for at least partially melting the polyester feedstock is an extruder, and the extruder is operated at a temperature of between 250 and 290° C.

19. The process according to claim 1, wherein each static or dynamic mixer is operated at a temperature of between 250 and 290° C., with a residence time of between 1 second and 5 minutes, the residence time being defined as the ratio between the volume of liquid in the static or dynamic mixer in relation to the sum of the volume flow rates of the polyester stream and of the fraction of the alcohol stream which feed the static or dynamic mixer under consideration.

20. The process according to claim 1, wherein conditioning step a) uses an extruder, followed by 2 to 4 static or dynamic mixers, placed in series, the extruder being fed with the polyester feedstock and with a fraction of the alcohol stream, such that the ratio by weight between said fraction of the alcohol stream, which feeds the extruder, and the polyester feedstock which feeds the extruder is between 0.003 and 0.050, the other fraction of the alcohol stream being divided into 2 to 4 partial streams of alcohol compound, the number of partial streams of alcohol compound being equal to the number of static or dynamic mixers used, each of the static or dynamic mixers being fed with a polyester stream and one of the partial streams of alcohol compound such that, in each static or dynamic mixer, the volume degree of dilution with alcohol compound is:
between 10% and 35%, when the ratio of the viscosities between the polyester stream and the partial stream of alcohol compound which feed the static or dynamic mixer under consideration is greater than or equal to 3000, or
between 20% and 65%, when the ratio of the viscosities between the polyester stream and the partial stream of alcohol compound which feed the static or dynamic mixer under consideration is less than 3000.
